(19) United States
(12) Patent Application Publication
(10) Pub. No.: US 2005/0220664 A1
(43) Pub. Date: Oct. 6, 2005

(54) Aroma cartridge and device for dispensing aroma substances, and control or regulation method

(75) Inventors: Alfred Hitzler, Mochenwangen (DE); Erich Harter, Mochenwangen (DE)

Correspondence Address: BURR & BROWN, PO BOX 7068, SYRACUSE, NY, 13261-7068, US

(73) Assignee: Venta-Duft GmbH, Weingarten (DE)

(21) Appl. No.: 11/085,836

(22) Filed: Mar. 21, 2005

Related U.S. Application Data
(63) Continuation of application No. PCT/DE03/03175, filed on Sep. 23, 2003.

(30) Foreign Application Priority Data
Sep. 23, 2002 (DE) 102 44 278.9
Aug. 14, 2003 (DE) 103 37 794.8

(51) Int. Cl. A61L 9/12

(52) U.S. Cl. 422/5; 422/124

(57) ABSTRACT

The invention relates to a aroma cartridge for a device for dispensing aroma substances, the aroma cartridge comprising at least one vessel having at least one cavity for receiving an aroma medium, the cavity having at least one opening, the opening being closed off by a metering device. Furthermore, the invention relates to the device for dispensing aroma substances from a aroma cartridge and to a method for operating the device.

### CROSS REFERENCE TO RELATED APPLICATIONS

This application is a continuation of International Application No. PCT/DE2003/003175, having an international filing date of Sep. 23, 2003, which designated the United States, the entirety of which is incorporated herein by. reference.

This application also claims the benefit of German Application No. 102 44 278.9, filed Sep. 23, 2002, and German Application No. 103 37 794.8, filed Aug. 14, 2003, the entireties of which are incorporated herein by reference.

### FIELD OF THE INVENTION

The invention relates to an aroma cartridge, to a device for dispensing aroma substances and to a method for controlling or regulating the device.

### BACKGROUND OF THE INVENTION

Devices of this type are used in particular to improve the air in rooms, for therapeutic purposes, and to carry out what is known as aromatherapy. The prior art has disclosed numerous devices for emitting aroma substances. For example, the subject matter of EP 0 004 039 B1 is a liquid atomizer in which the liquid that is to be atomized is delivered via a wick, using the capillary action, to an atomizer plate, and the atomizer plate is excited by a vibration generator. German utility model DE 296 21 752 U1 has disclosed, for example, a device for the distribution of aroma substances in which a fan blows onto a distributor mat, which is impregnated from a tank, with a solenoid pump delivering the oil through a cannula from the tank to the distributor mat. One drawback of the prior art is that the known solutions either require a technically complex pump mechanism or rely on delivery by means of capillary action. Delivery with the aid of capillary action does not allow the delivery to be controlled, since delivery is constant. This leads to distribution of aroma or consumption of aroma substance even when use of the device is not desired. Furthermore, U.S. Pat. No. 4,200,229 has disclosed an aroma distributor, which comprises a stationary holder and a replaceable aroma cartridge. Delivery of the liquid into a cushion is, in this case, effected by a manually actuated pump. A drawback of this is that lines with a small internal diameter have the tendency to become blocked. A drawback of all these devices is that they do not allow the release of aroma to the environment to be effectively switched off, since the impregnated carriers generally continue to release aroma to the environment even after any fan or other distributor that is present has been switched off.

### SUMMARY OF THE INVENTION

It is an object of the invention to propose an aroma cartridge and a device for dispensing aroma substances, as well as a method for controlling or regulating the device, which, by virtue of a simple structure and simple procedure, allows reliable and controllable transfer of the aroma medium to the material which releases the aroma substance, and which also allows the dispensing of aroma to be effectively stopped within a very short time.

The invention relates to an aroma cartridge for a device for dispensing aroma substances, the aroma cartridge comprising at least one vessel which has at least one cavity for receiving an aroma medium, which cavity opens toward the environment, with a metering device closing off the opening; in this aroma cartridge, it is provided in particular that the metering device has a plunger which can be displaced into a first and a second limit position through the opening in the vessel, the limit positions lying opposite one another and the plunger, in the first limit position, being at least partially in an impregnation position within the vessel, and the plunger, in the second limit position, being in an aroma-dispensing position in which it is at least partially outside the vessel. With an aroma cartridge that is equipped with a metering device of this type or a metering valve of this type, it is possible for the liquid or gaseous aroma medium to be carried out of the vessel into the environment without any risk of flexible tubes with a small diameter becoming blocked. The plunger forms a carrier medium used to temporarily store the aroma medium. Since the plunger can be displaced back into the cavity that is closed off with respect to the environment, the dispensing of aroma can be switched off quickly. This is because the plunger, which releases the aroma, is encapsulated inside the cavity after it has been retracted.

An advantageous development of the subject matter of the invention provides for the plunger to be assigned at least one seal and in particular two seals. This makes it possible to seal off the plunger or cavity with respect to the environment in the first and second limit positions, preventing the aroma medium from escaping from the aroma cartridge. In particular, there is provision to use seals made from Teflon, since such seals are particularly resistant to aggressive aroma media.

According to a particular embodiment of the subject matter of the invention, there is provision for the cavity to be sealed off from the outside by means of the first seal and for the cavity to be sealed off from the inside by means of the second seal. This makes the metering device of particularly simple construction, allowing simple and inexpensive assembly of the metering device.

The invention provides for the use of a plunger, which allows temporary storage of the aroma medium in at least a portion thereof. This allows the volume of the store and its position on the plunger to be selected as desired such that it can easily be matched to the prevailing requirements.

One particular embodiment provides for at least one cavity to be formed in the plunger, where the cavity is closed off by a grid and holds a material, which is suitable for storing the aroma medium. In a cage of this type, it is possible to use storage bodies or impregnation bodies with a very wide range of properties, e.g. different pore sizes, without the design of the plunger having to be modified.

The invention provides for, in particular, open-cell plastics or foams, in particular formed from polypropylene or polyethylene, or open-cell ceramic elements, or Teflon meshes to be used as materials for the storage body, with the meshes being, for example, wound up in order to form a storage body. Sponge-like storage bodies with a structure of this type allow storage of the preferably concentrated essential or natural oils used as aroma medium.

Furthermore, the invention provides for the metering device to be equipped with a spring element, which presses the plunger into the first limit position. This ensures that, in the as-delivered state of the aroma cartridge or in the at-rest position of the aroma cartridge, the plunger is always in a position in which it is retracted as far as possible into the cavity and in which the cavity is sealed off with respect to the environment. This effectively prevents the undesired escape of aroma medium.

According to the invention, there is provision for the metering device to be constructed from a closure cap in which the plunger is guided. This makes it possible to design the metering device as a structural unit, which can be preassembled and merely has to be fitted onto the container in order to form a functioning aroma cartridge.

A particularly advantageous embodiment of the invention provides for the closure cap and the vessel to be provided with a screw thread in order to allow the closure cap to be screwed onto the vessel. An aroma cartridge assembled in this way allows economic production and is also optimally suited to subsequent separation of the materials of value or for refilling.

Furthermore, it is provided for at least one guide element, into which the plunger is guided, to be arranged at the closure cap. This supporting feature for the plunger limits the extent to which the latter can be subject to load from laterally acting forces as it passes through the closure cap. This effectively prevents leaks which could be caused by transverse loads or the plunger being in sloping positions. Furthermore, the guidance assists with uniform, gentle sliding of the plunger between the limit positions.

According to the invention, guide elements are provided, which are designed as a rail or a bar. By way of example, such guide elements can easily be molded onto the closure cap during the injection-molding process.

The invention also provides for the vessel to be formed from glass, such as a glass bottle, since aroma media often contain aggressive substances.

It is advantageous for the aroma cartridge to be provided, at the vessel and/or the metering device, with at least one projection and/or recess. This allows simple handling of the aroma cartridge or its individual parts during the production process and during the filling operation. Furthermore, this also allows the aroma cartridge to be arranged in an accurate position in the device.

According to a variant embodiment, there is provision for the plunger to be of at least a two-part design. In the case of a plunger with stops or of a plunger with seals, which protrude beyond the periphery of the plunger, this allows simple mounting of the plunger on the closure cap, since the plunger can be introduced into a through-bore in the closure cap from two sides.

The invention also provides for the seal to be designed as a sealing disk. This shape allows simple fitting of the seals to the plunger. Furthermore, these seals allow simple monitoring of their function.

According to a variant embodiment, the invention provides for the use of plunger ring seals as seals. These seals also allow the plunger to be sealed off with respect to the vessel or the closure cap during the movement between the limit positions.

A third variant embodiment provides for the seal to be designed in gland form, the gland being mounted in the vessel or in the closure cap. If a gland is used, a single seal is sufficient to seal off the plunger or the cavity with respect to the environment.

According to the invention, it is also provided that at least one stop be arranged at the plunger. This prevents the plunger from being displaced undesirably far beyond a limit position. In particular, it is provided that the stop butts against the vessel and/or against the closure cap.

Furthermore, the invention relates to a device for dispensing aroma substances, having an aroma cartridge as described above which is held releasably in the device. In this device, it is provided that the plunger of the aroma cartridge be moved from the first limit position into the second limit position and from the second limit position into the first limit position by a first control means of the device. It is possible to switch the aroma cartridge on and off by means of this simple sequence of movements. The time required to displace the plunger is generally all that it takes to effect the switching on or off. As a result of being switched off, the plunger is retracted into the vessel, in which it is held in a sealed manner so that no further aroma is dispensed from the plunger.

An advantageous development of the subject matter of the invention provides for the aroma cartridge to be rotated by a second control means, in order for the plunger or the material arranged in or at the plunger to be completely impregnated. This allows even short plungers to be used and the aroma medium contained in the aroma cartridge to be used in full for spreading the aroma, without residual quantities having to be left in the aroma cartridge.

Furthermore, the invention provides for the first and/or second control means to be controlled with the aid of a third control means. This makes it possible to maintain a predefined sequence of movements and prevents incorrect control sequences with respect to independent actuation of the first and second control means.

According to the invention, the control means are designed as mechanical and/or electromechanical and/or electronic control means. Mechanical control means provided are, in particular, levers, slot guides, tensioning means and gearwheels. As electromechanical control means, the invention provides, in particular, electric motors, pneumatic cylinders and electromagnets. According to the invention, electronic control means are intended, in particular, to be integrated circuits.

Furthermore, there is provision for the control means to be regulated by a higher-level electronic regulation system or for the control means to be combined with an electronic regulation system. This ensures that the individual components interact in the planned way and/or interact with further components.

In addition, there is provision for at least one sensor to be connected to the regulation system. This makes it possible to react to further parameters during regulation.

An advantageous refinement of the subject matter of the invention provides for the device to be assigned at least one fan, by which the plunger can be exposed to a flow of air in the aroma-dispensing position. This allows the aroma to be distributed through a wide area.

According to the invention, stepped or continuous regulation of the power of the fan is also provided. This makes it possible to match up with individual conditions and requirements.

Finally, the invention relates to a method for controlling or regulating the device described above for dispensing aroma substances, comprising the following steps: firstly, rotating the aroma cartridge out of an upright basic position, in which the plunger is approximately vertical, through approximately 90° to 180° into a loading position, in which the plunger is then in the first limit position; secondly, holding the aroma cartridge in the loading position in order to effect impregnation of the material which is able to take up liquid; thirdly, pivoting the aroma cartridge back into the basic position; fourthly, raising the plunger into the second limit position; fifthly, directing an air flow onto the plunger in order to dispense aroma substances, and sixthly, moving the plunger back into the first limit position. A method of this type allows repeated, controllable introduction of aroma into an environment.

Alternatively, a further method for controlling or regulating the device described above for dispensing aroma substances is provided, comprising the following steps: firstly, raising the plunger out of the first limit position into the second limit position; secondly, directing an air flow onto the plunger in order to dispense aroma substances, and thirdly, moving the plunger back into the first limit position. A method of this type likewise allows repeated, controllable dispensing of aroma into an environment.

### BRIEF DESCRIPTION OF THE DRAWINGS

Further details of the invention are described in the drawing on the basis of diagrammatically depicted exemplary embodiments. In the drawing:

FIGS. 1a-1c show a first, diagrammatically depicted aroma cartridge in various operating positions;

FIGS. 2a-2c show a second diagrammatically depicted aroma cartridge in various operating positions;

FIG. 3a shows a third diagrammatically depicted aroma cartridge;

FIG. 3b diagrammatically depicts a device for operating the aroma cartridge shown in FIG. 3a;

FIGS. 3c-3e diagrammatically depict the device with inserted cartridge in various operating positions and views;

FIGS. 4a-4n show a number of different illustrations of a fourth aroma cartridge;

FIGS. 5a-5j show a device for operating the aroma cartridge illustrated in FIGS. 4a to 4n in various operating positions and views, and

FIG. 6 diagrammatically depicts a further device with aroma cartridge.

### DETAILED DESCRIPTION OF THE INVENTION

Surfaces taken in section are not generally illustrated in hatched forms in the figures, in order to maintain clarity of the figures.

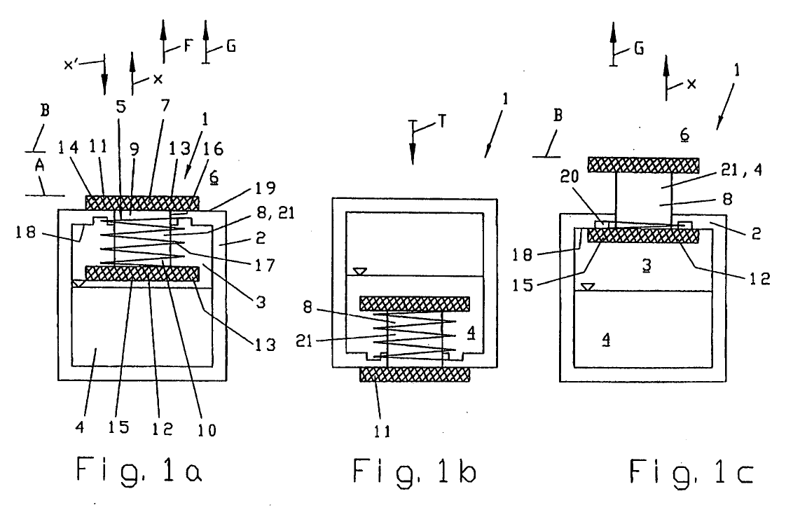

FIGS. 1a to 1c diagrammatically depict a first aroma cartridge 1 in various operating positions. First of all, a thorough description of the aroma cartridge 1 will be given with reference to FIG. 1a. The aroma cartridge 1 comprises a vessel 2 having a cavity 3, the cavity 3 holding a liquid aroma medium 4. The vessel 2 has an opening 5, by means of which the cavity 3 is open toward the environment 6. The opening 5 is closed off by a metering device 7. The metering device 7 comprises a plunger 8, which at each of its end-side ends 9 and 10 has a seal 11 and 12, respectively. The seals 11 and 12 are designed as sealing disks 13 and form stops 14 and 15. The opening 5 in the vessel 8 forms a sliding guide 16 for the plunger 8, in which the latter can move in arrow directions x and x′. The plunger 8 can be displaced out of a first limit position A, illustrated in FIG. 1a, into a second limit position B, illustrated in FIG. 1c, in arrow direction x. Since the plunger 8 is pressed into limit position A by a spring element 17, a force F is required for the displacement into the second limit position B. The spring element 17, which is designed as a coil spring 17, is supported against an inner wall 18 of the vessel 2 and acts on the plunger 8 via the second seal 12. In the first limit position A, the first seal 11 forms the stop 14 and seals off the interior 3 of the vessel 2 with respect to the environment 6 from the outside. In this case, the stop 14 bears against an outer wall 19 of the vessel 2 and prevents the plunger 8 from being pulled further into the cavity 3 by the spring element 17. In the second limit position B of the plunger 8 (cf. FIG. 1c), the second seal 12 forms the stop 15, which bears against the inner wall 18 of the vessel 2 and prevents the plunger 8 from being pulled out further in the arrow direction x. In the second limit position B, the spring element 17 is fully compressed and is located in an annular recess 20 in the vessel 2. Furthermore, the seal 12 seals off the interior 3 of the vessel 2 with respect to the environment 6. The plunger 8 consists of a material 21, for example a porous ceramic body, which is suitable for taking up the aroma medium 4. In principle, the following procedure is provided for the dispensing of aroma to the environment 6: the aroma cartridge 1 is turned from an upright basic position G (cf. FIG. 1a) for approximately 180° into an impregnation position T (cf. FIG. 1b), with the plunger 8 in the first position A, i.e. a plunger impregnation position A. In the impregnation position T of the aroma cartridge 1, the plunger 8 or the impregnatable material 21 which forms the plunger is completely surrounded by the aroma medium 4. The aroma medium 4 is prevented from escaping in the impregnation position T by the first seal 11. After impregnation, the aroma cartridge 1 is rotated back out of the impregnation position T into the basic position G, for approximately 180°, and the plunger 8 is raised in arrow direction x. In this second limit position B of the plunger 8, which is an aroma-dispensing position B, the aroma medium 4 which has been taken up by the plunger 8 is dispensed to the environment 6. As a result of the plunger 8 being released, it springs back into the first limit position A and the aroma-releasing operation is at an end, with further release of aroma to the environment 6 by the plunger 8 being prevented. In the first limit position A, the plunger 8 is once again completely inside the interior 3 of the vessel 2, so that it is possible to describe the aroma cartridge 1 as having been switched off. When using a gaseous aroma medium 4, it is not generally necessary for the aroma cartridge 1 to adopt the impregnation position T, since the plunger is already completely surrounded by the aroma medium 4 in the basic position G of the aroma cartridge and can therefore take this up.

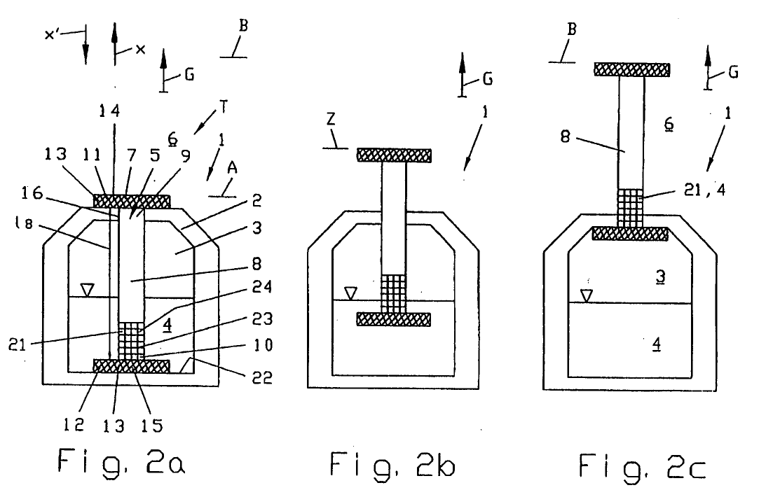

FIGS. 2a to 2c diagrammatically depict a second aroma cartridge 1 in various operating positions. The description above in connection with FIGS. 1a to 1c in principle also applies to FIGS. 2a to 2c. Unlike in the case of the aroma cartridge illustrated in FIGS. 1a to 1c, FIGS. 2a to 2c show an aroma cartridge 1 which has a plunger 8 that in the first limit position A, on account of its length 1₈, extends all the way to a base 22 of the vessel 2. Therefore, in contradiction to the aroma cartridge illustrated in FIGS. 1a to 1c, there is no difference between a basic position G of the aroma cartridge 1 and an impregnation position T of the aroma cartridge 1, since there is no need to turn the aroma cartridge 1. Consequently, the aroma cartridge 1 is simultaneously in positions G and T. Furthermore, the plunger 8 is not continuously made from a material for taking up the aroma medium 4, but rather has a cage 24 which is delimited by a grate 23 and is filled with material 21 for taking up the aroma medium 4. To dispense the aroma medium 4 stored in the material 21, the plunger is raised from the first limit position A (cf. FIG. 2a), via an intermediate position Z (cf. FIG. 2b), into the second limit position B (cf. FIG. 2c), counter to the force of a spring element, which is not illustrated in FIGS. 2a to 2c. In the second limit position B, the plunger 8 or the material 21 arranged in the cage 24 dispenses the aroma substance 4, which it has taken up to the environment 6. The aroma cartridge 1 is “switched off” by the plunger 8 being retracted into the first limit position A, which is illustrated in FIG. 2a. Contrary to the sequence shown in FIGS. 1a to 1c, the aroma cartridge 1 shown in FIGS. 2a to 2c is in the basic position G during all the steps; in this case, there is no need to turn the aroma cartridge 1.

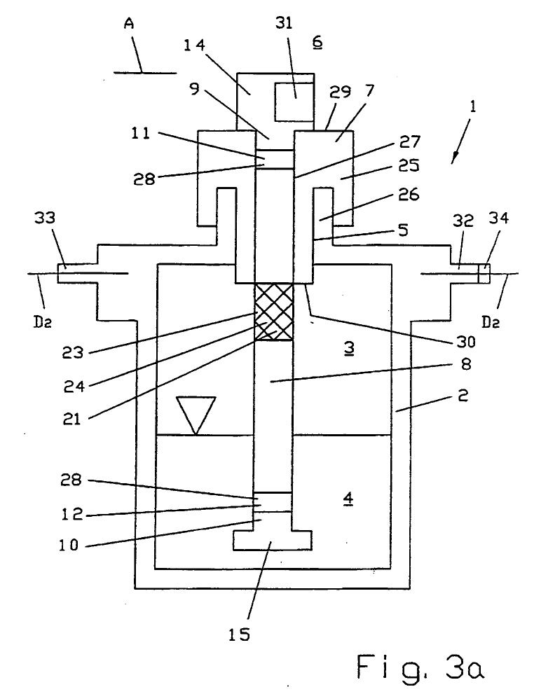

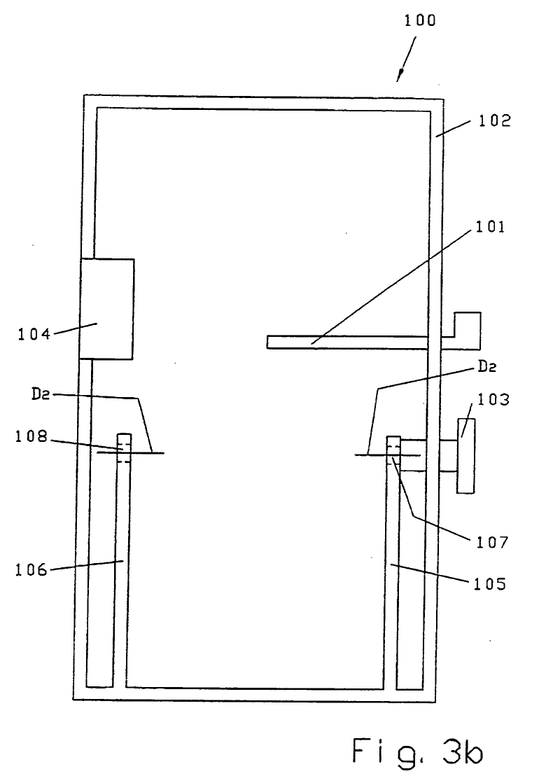

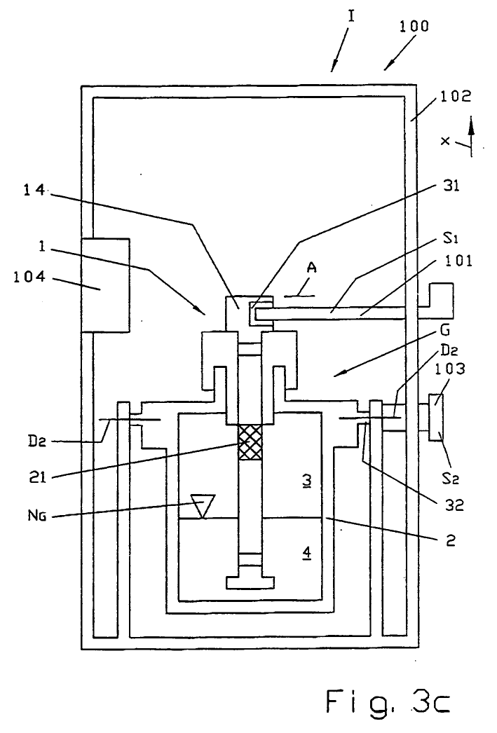

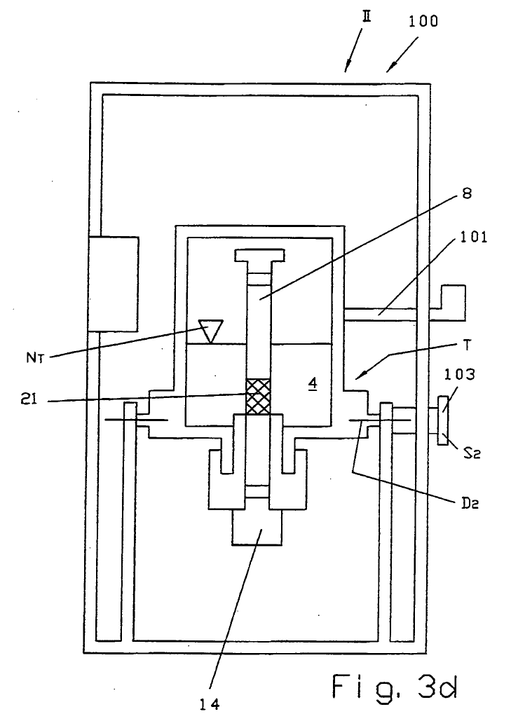

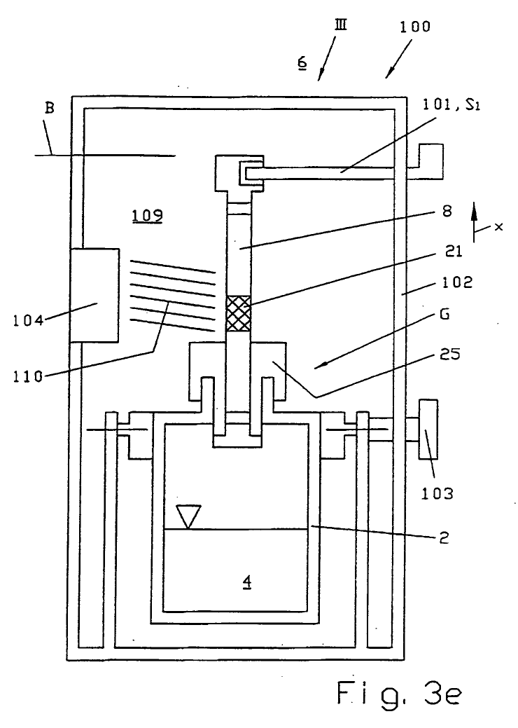

FIG. 3a diagrammatically depicts a third aroma cartridge 1. The description given in connection with FIGS. 1a to 1c in principle also applies to FIG. 3a. FIG. 3a shows an aroma cartridge 1 with a different form of metering device 7 compared to the aroma cartridge illustrated in FIGS. 1a to 1c. The metering device 7 comprises a closure cap 25, which is screwed to a neck 26 of the vessel 2 (the screw threads are not illustrated) and projects through the opening 5 in the vessel 2 into the interior 3 of the latter. The plunger 8 is mounted in a cylindrical passage 27 in the closure cap 25. The plunger 8 or the interior 3 is sealed off with respect to the closure cap 25 or the environment 6 by means of the two seals 11 and 12, which are designed as plunger ring seals 28. Stops 14 and 15, which in the limit positions A and B of the plunger 8 (cf. also FIG. 3e) butt against a top side 29 and an underside 30, respectively, of the closure cap 25, are formed at the end-side ends 9 and 10 of the plunger 8. A pocket 31, into which a lever 101 (cf. for example FIG. 3b or 3c) can be introduced, is formed at the upper stop 14. Furthermore, the plunger 8 does not completely consist of a material for taking up the aroma medium 4, but rather has a cage 24 that is delimited by a grid 23 and holds material 21 which is suitable for temporarily storing the medium 4. Journals 32 and 33, which are provided for the purpose of suspending the aroma cartridge 1 such that it can rotate about an axis of rotation D₂, are formed integrally on the vessel 2 of the aroma cartridge 1. The journal 32 has a flattened portion 34, thereby allowing the journal 32 to be coupled to a rotary button 103 in a manner fixed in terms of rotation (cf. FIG. 3b).

FIG. 3b shows a device 100 for dispensing aroma substances into which the aroma cartridge 1 shown in FIG. 3a can be inserted. The device 100 substantially comprises a housing 102 at which the abovementioned lever 101, the abovementioned rotary button 103, and a fan 104 are mounted. Furthermore, two limbs 105 and 106, which have bores 107 and 108 for mounting the journals of the aroma cartridge about axes of rotation D₂, are formed integrally on the housing 102.

FIG. 3c shows the device 100 illustrated in FIG. 3b with the aroma cartridge 1 illustrated in FIG. 3a having been inserted in the device 100. The aroma cartridge 1 is in this figure in a basic position G, with the device 100 in an at-rest position I. The lever 101 forms first control means S₁, which can be used to raise the plunger 8 out of the first limit position A into the second limit position B (cf. FIG. 3e). For this purpose, the lever 101 engages in the pocket 31 in the stop 14 and is moved upward in arrow direction x. The pocket 31 is designed to be open toward the person viewing the figure. The rotary button 103 forms second control means S₂ which allow the aroma cartridge 1 to be rotated about the axis of rotation D₂. For this purpose, a plug connection which is fixed in terms of rotation is formed between the rotary button 103 and the journal 32, which is designed as an extension of the vessel 2. The aroma medium 4 is at a level N_{G} in the interior 3 of the vessel 2. The material 21 mounted in the plunger 8 for taking up the aroma medium 4 is located above the level N_{G} of the aroma medium 4.

FIG. 3d shows the device 100 illustrated in FIG. 3c in a filling position II, with the aroma cartridge 1 in an impregnation position T. The aroma cartridge 1 is transferred from the basic position G shown in FIG. 3c into the impregnation position T shown in FIG. 3d by the rotary head 103 being rotated through 180° about the axis of rotation D₂. Rotation of the aroma cartridge 1 disconnects the stop 14 and the lever 101 from one another. In the impregnation position T, the material 21 for taking up the aroma medium 4 is fully in the aroma medium 4, which fills the aroma cartridge 1 up to a level N_{T} in the impregnation position T. After impregnation, the aroma cartridge 1 is then rotated back into the basic position G shown in FIG. 3c by means of the rotary button 103 or the second control means S₂. In the process, the lever 101 engages in stop 14 once again. Therefore, after this step of rotating back, the stop 14 or the plunger 8 can be displaced out of the first limit position A into the second limit position B by the lever 101 or the first control means S₁ (cf. FIG. 3e).

FIG. 3e shows the device 100 in the aroma position III described above, in which the aroma cartridge 1 is held in the basic position G and the plunger 8 with the freshly impregnated material 21 has been displaced into its second limit position B. For this purpose, the lever 101 or the first control means S₁ was moved in the arrow direction x at the housing 102. In the second limit position B of the plunger 8, the plunger 8 has been extended out of the vessel 2 or the closure cap 25 to such an extent that the material 21 that has been impregnated with the aroma medium 4 is located in a freely accessible position outside the vessel 2 and outside the closure cap 25, in an interior space 109 of the device 100. It is preferable for the material 21 or the plunger 8 to be exposed to an air flow 110 from the fan 104 in order at least from time to time to rapidly distribute the aroma medium in the environment 6 outside the housing 102. For this purpose, the housing 102 has openings (not shown) which allow air to be exchanged between the interior 109 and the environment 6. The distribution of aroma to the environment 6 is effected by switching off the fan 104 and retracting the plunger 8 into the first limit position A with the aid of the lever 101. The device 100 is then once again in the at-rest state shown in FIG. 3c.

According to a variant embodiment (not shown), there is provision for the first and second control means to be designed as electromechanical control means, in each case comprising an electric motor and mechanical components. A third control means, designed, for example, as control electronics which simultaneously also control operation of the fan, is provided for the purpose of controlling the first and second control means.

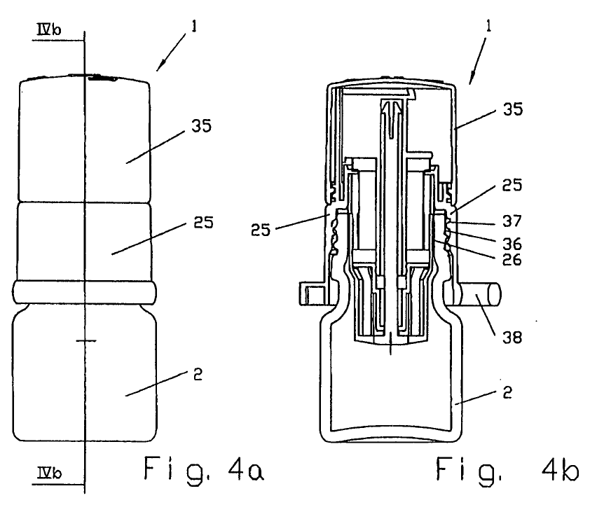

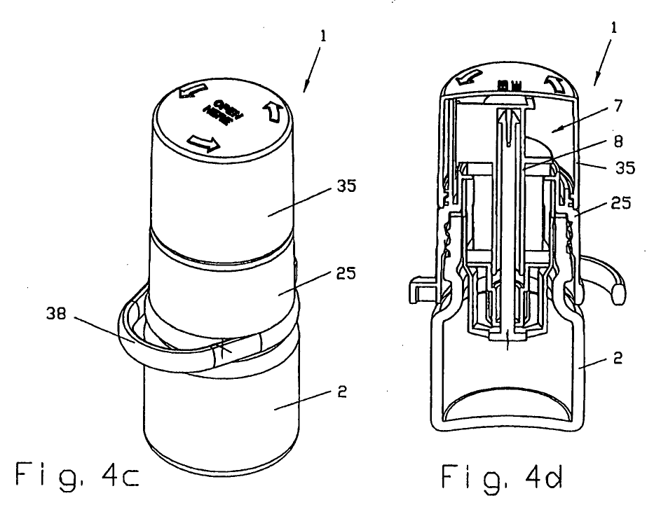

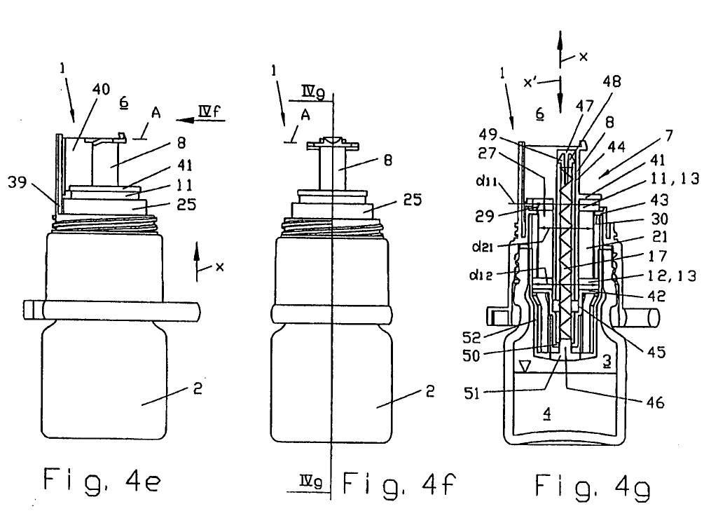

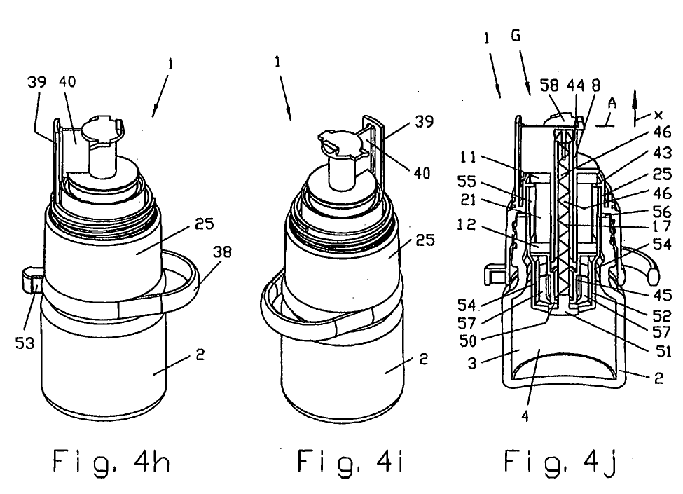

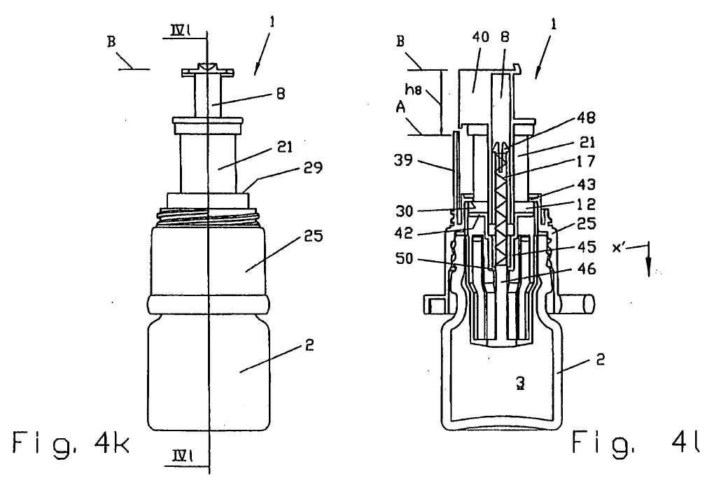

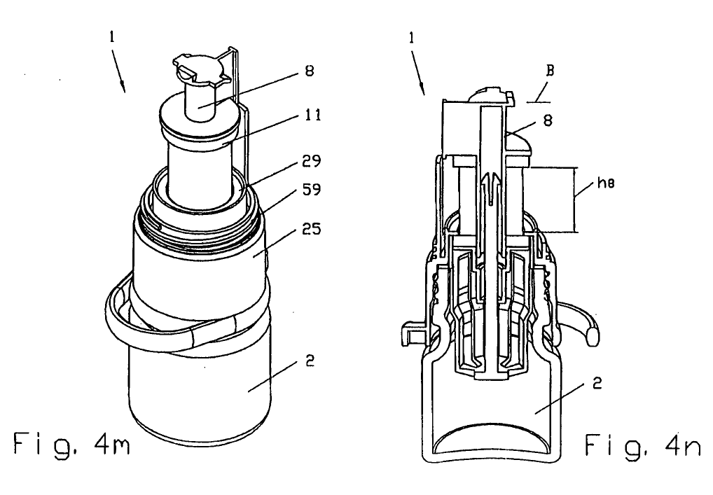

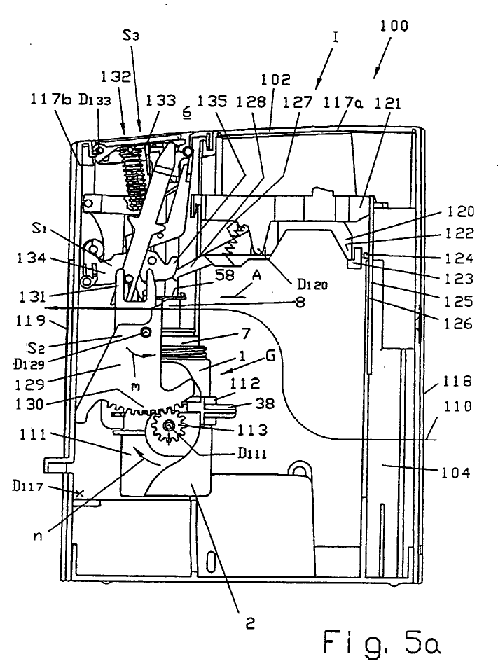

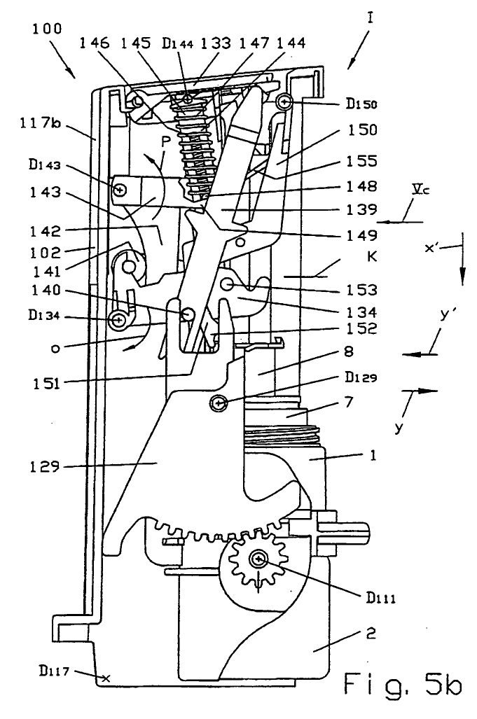

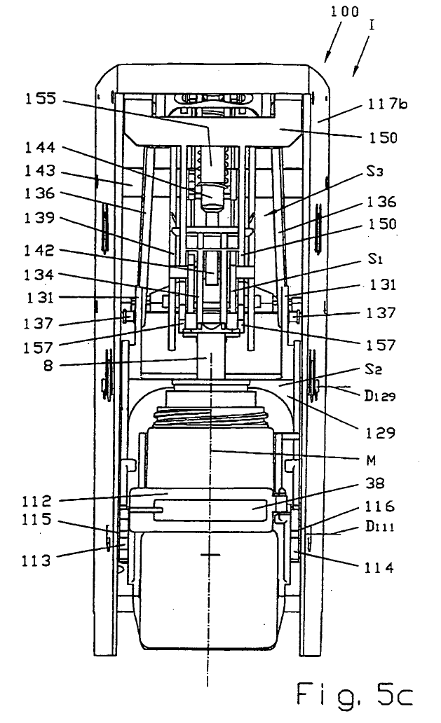

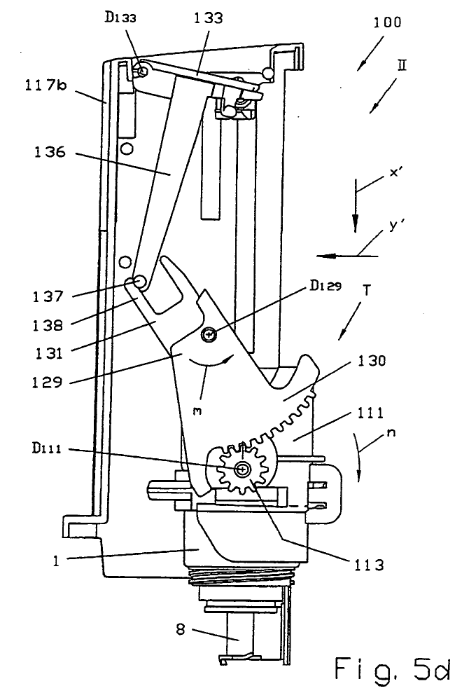

FIGS. 4a to 4n show a fourth embodiment of an aroma cartridge 1 in the form of various different illustrations. FIG. 4a shows a side view of the aroma cartridge 1. This view shows a vessel 2, a closure cap 25 and a cover 35 for the closure cap 25 of the aroma cartridge 1.

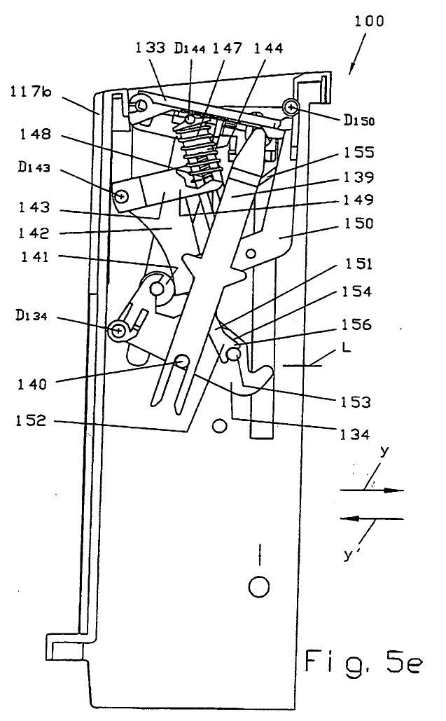

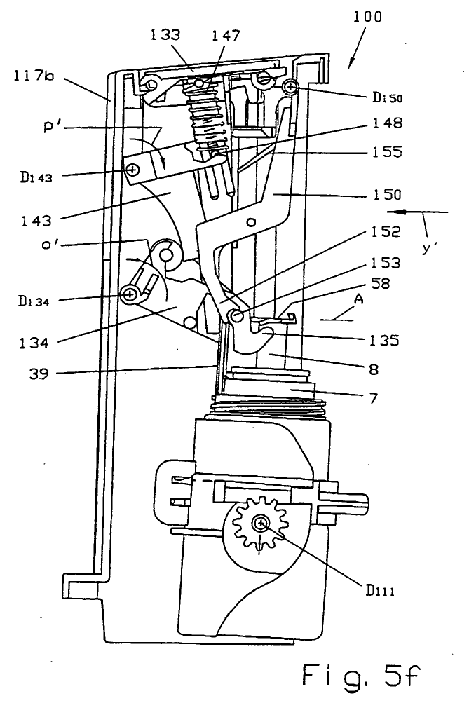

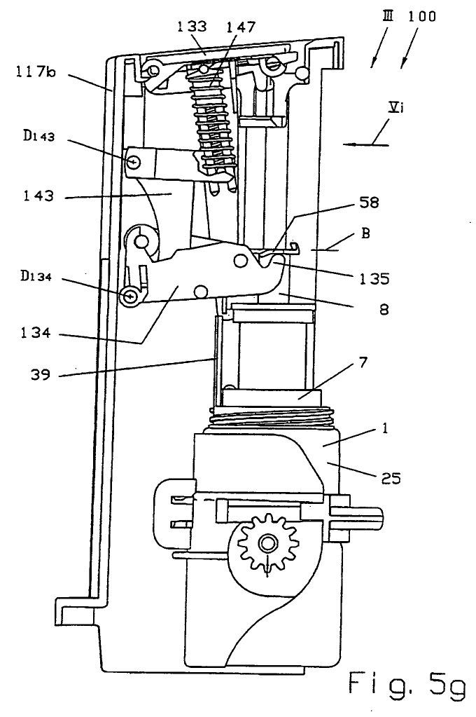

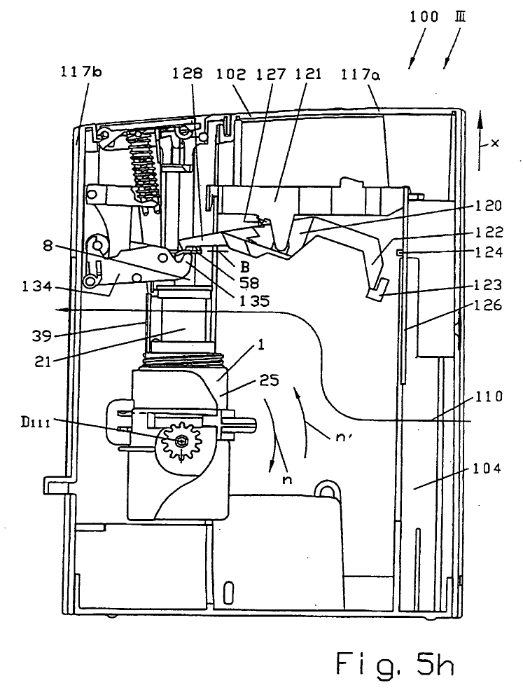

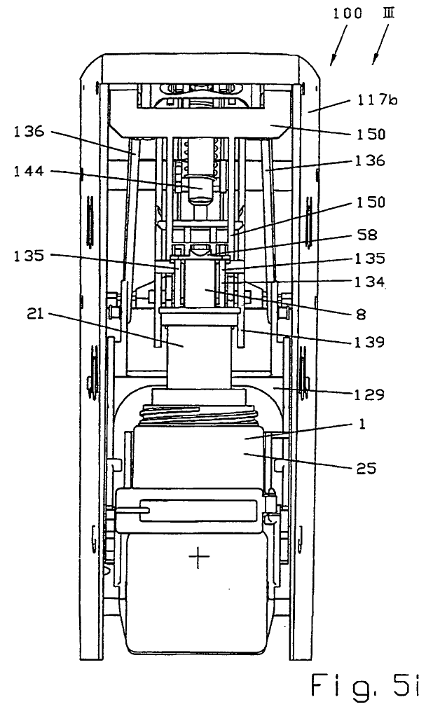

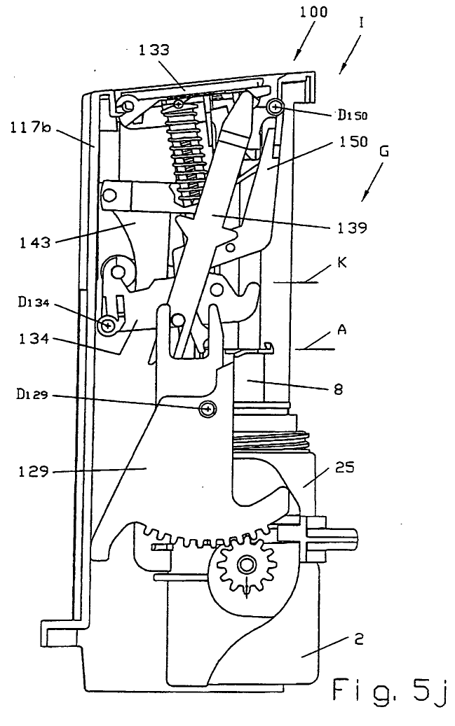

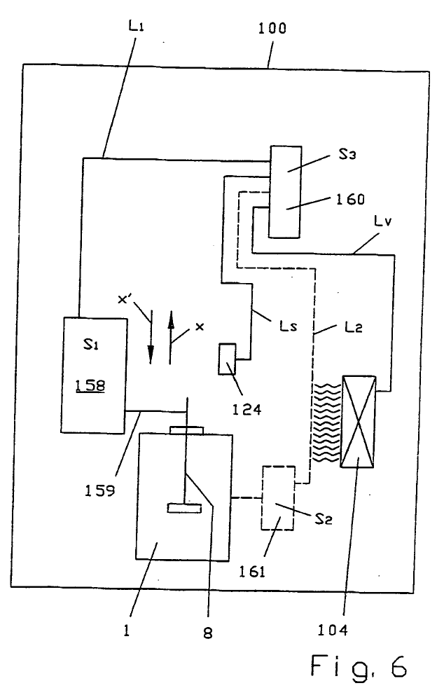

FIG. 4b shows a section through FIG. 4a on section line IVb-IVb. The vessel 2 has a neck 26 with an external screw thread 36, onto which the closure cap 25, which has an internal screw thread 37, is screwed. A clip 38, which is used to fix the aroma cartridge 1 in the correct position in a device 100 (cf. for example FIG. 5c), is formed integrally on the closure cap 25.

FIG. 4c shows a perspective illustration of the aroma cartridge 1 which has already been shown in FIG. 4a. This figure reveals how the clip 38 surrounds the closure cap 25 in the form of a bead.

FIG. 4d shows a perspective illustration of the section indicated in FIG. 4a. The closure cap 25 and a plunger 8 together with further components form a metering device 7 that can be screwed onto the vessel 2.

FIG. 4e shows a side view of the aroma cartridge 1 with the cover unscrewed. The plunger 8 is visible above the closure cap 25. A guide element 39, which extends in an arrow direction x and forms a guide with a T-groove is formed at the closure cap 25. The plunger 8 is guided in this T-groove of the guide element 39 by means of a web 40 that is T-shaped in cross section, in order to prevent the plunger from tilting in the event of an up-and-down movement out of the first limit position A into the second limit position B (cf. for example FIG. 4k) as a result of laterally acting forces. The web 40 itself is stabilized by a first disk 41, beneath which a first seal 11 is located. The seal 11 closes off the vessel 2 in the first limit position A, shown in FIG. 4e, of the plunger 8.

FIG. 4f shows a view of the aroma cartridge 1 from arrow direction IVf illustrated in FIG. 4e. The web 40 is covered in this view.

FIG. 4g shows a section through FIG. 4f on section line IVg-IVg. In addition to the upper disk 41 and the first seal 11, FIG. 4g also reveals a lower disk 42 and the second seal 12, which belong to the plunger 8. The material 21 for taking up the aroma medium 4 is arranged between the seals 11 and 12 in an interior 3 of the vessel 2. This is approximately in the form of a hollow cylinder, with the diameter d₂₁ of the impregnation body 21 being smaller than the diameters d₁₁ and d₁₂ of the seals 11 and 12. It is therefore possible for the impregnation body to pass through the cylindrical passage 27 in the closure cap 25 in arrow directions x and x′The cylindrical passage 27 is formed by a shoulder 43 of the closure cap 25. A top side 29 of the shoulder 43 forms a bearing surface 29 for the first seal 11, and an underside 30 of the shoulder 43 forms a bearing surface 30 for the second seal 12. The plunger 8, which can be displaced in arrow directions x and x′ in the closure cap 25, therefore substantially comprises a lifting tube 44, on which the web 40 and the disk 41 are formed integrally, and the second disk 42, which is formed by a cap 45 fitted onto the lifting tube 44. The plunger 8 also includes the sealing rings 13, which hold the impregnation body 21 between them. The metering device 7 is completed by a pin 46, which runs in a cylindrical interior 47 of the lifting tube and has a mushroom-shaped first end 48. The first end 48 forms a first abutment 49 for a spring element 17. A second abutment 50 for the spring element 17 is formed by the cap 45. A second end 51 of the pin 46 is designed as a basket 52, which is supported from below against the shoulder 43 of the closure cap 25. According to an embodiment, which is not illustrated, the pin and the basket are of a two-part design and are preferably held together by means of a latching connection.

FIGS. 4h to 4j show a perspective view of FIGS. 4e to 4g. It can be seen from FIG. 4h that in the region of the clip 38 the closure cap 25 has a projection 53, which is intended to engage in a positively locking manner in the device 100.

FIG. 4i clearly illustrates the T-shaped cross section of the web 40, without the T-groove formed in the guide element 39 being illustrated in detail, in order to simplify the figures.

FIG. 4j clearly shows the basket 52 formed integrally at the second end 51 of the pin 46. The basket is substantially formed by arms 54, which merge into a ring 55 that bears against the closure cap 25 and is supported against a shoulder 43 of the closure cap 25. A seal 56 is arranged between the vessel 2 and the closure cap 25 in order to seal off the two components with respect to one another. Raising of the plunger 8 or the lifting tube 44 out of the first limit position A, illustrated in FIGS. 4a to 4j, also moves within the cap 45, which is fixedly connected to the lifting tube 44, and therefore the seals 11 and 12 and the impregnation body 21. The raising of the plunger 8 and of the components fixedly connected to it takes place counter to the force of the spring element 17. As the plunger 8 is raised in arrow direction x, the spring elements 17 is compressed to an increasing extent by the abutment 50 formed by the cap 45. The pin 46 remains in an unchanged position, since it is supported against the closure cap 25 by means of the basket 52. Therefore, the plunger 8 forms a moveable part of the metering device 7, and the pin 46, together with the basket 52, forms a stationary part of the metering device 7. The impregnation body 21 is generally impregnated before the plunger 8 is raised. For this purpose, the liquid 4 that is present in the interior 3 of the vessel 2 can advance to the impregnation body 21 through gaps 57 which are formed between the arms 54 of the basket 52. In the embodiment of the aroma cartridge 1 illustrated in FIGS. 4a to 4n, the impregnation takes place in particular when the aroma cartridge 1 has been pivoted through approximately 90° to 180° out of the upright position G shown. To raise the plunger 8, a device 100 engages (cf. for example FIG. 5h) in particular on a cover 58 of the lifting tube 44.

FIG. 4k shows a side view of the metering cartridge 1, with the plunger 8 here in the second limit position B. This illustration should in particular be compared with FIG. 4f, which shows the metering cartridge in the same view but in the first limit position A of the plunger 8. In the second limit position B, the plunger 8 has been extended from the vessel 2 or the closure cap 25 sufficiently far for the impregnation body 21 to be virtually completely projecting above the top side 29 of the closure cap 25.

FIG. 4l shows a section through the aroma cartridge 1 illustrated in FIG. 4k on section line IVl-IVl. In the second limit position B of the plunger 8, the pin 46 is in an unchanged position with respect to the vessel 2 or with respect to the closure cap 25. The spring element 17 is now more compressed between the mushroom-shaped end 48 of the pin 46 and the abutment 50 formed by the cap 45. The interior 3 of the vessel 2 is sealed off in position B of the plunger 8 by the second seal 12, which is pressed by the disk 42 onto the underside 30 of the annular shoulder 43 of the closure cap 25. The compressed spring element 17 presses the plunger 8 back out of the second limit position B into the first limit position A, with the spring element 17 in the process being relieved of stress by the abutment 50 migrating back into the vessel 2 in direction x′. In the first limit position A too (cf. for example FIG. 4g), the spring element 17 still presses the plunger 8 in arrow direction x′ by way of the abutment 50. This ensures that the seal 11 bears with pressure against the closure cap 25 and in the first limit position A good sealing of the interior 3 with respect to the environment 6 is produced. As an alternative to the illustration presented in FIG. 4l, the invention provides, according to a variant embodiment, for the web 40 still to be guided in the guide element even in limit position B. This is achieved, for example, by a lengthened guide element or a reduced displacement of the plunger.

FIGS. 4k and 4l are illustrated once again in perspective in FIGS. 4m and 4n. FIG. 4m shows the bearing surface 29, on which the seal 11 comes to bear in the first limit position A of the plunger 8. The closure cap 25 has a screw thread 59, which is intended for the cover 35 to be screwed onto (cf. FIG. 4a).

FIG. 4n also illustrates a displacement h₈ covered by the plunger 8 between the first limit position A and the second limit position B. The spring element 17 has not been illustrated in some of FIGS. 4a to 4n, in order to maintain clarity within the figures.

FIGS. 5a to 5j show the above-mentioned device 100 for operating the aroma cartridge 1 illustrated in FIGS. 4a to 4n.

FIG. 5a shows a side view of the device 100, illustrating a sectional view through a housing 102. The housing 102 substantially comprises a first housing part 117a and a second housing part 117b. The aroma cartridge 1 is held in a positively locking manner in a holder 111, with an arm 112 engaging over the clip 38 of the aroma cartridge 1. Gearwheels 113 and 114 are connected in a rotationally fixed manner to the holder 111 (cf. also FIG. 5c). Journals 115 and 116, which are mounted in bores (not illustrated) in the second housing part 117b such that they can rotate about an axis of rotation D₁₁₁, are formed integrally at the gearwheels 113 and 114. The housing part 117b can be pivoted out of the housing part 117a, together with the holder 111 for the aroma cartridge 1, about an axis of rotation D₁₁₇. As a result, the aroma cartridge 1 is readily accessible and consequently easy to exchange. A fan 104 is arranged in the housing 102 and sucks in ambient air through openings (not shown) at a rear side 118 of the housing 102 and blows this air into the environment 6 through openings (not shown) at a front side 119 of the housing 102. The plunger 8 of the aroma cartridge 1 is positioned in this air flow 110. However, in FIG. 5a the plunger 8 is in the first limit position A, in which it is not intended to dispense aroma by means of the metering device 7. Basic operational readiness of the fan 104 is controlled by means of a lever 120, which is arranged at a bridge 121 such that it can rotate about an axis of rotation D₁₂₀. At a first end 122, the lever 120 bears a permanent magnet 123. The permanent magnet 123 interacts with a sensor 124 arranged on a printed circuit board 125. The first lever 120 adopts the position illustrated in FIG. 5a when the aroma cartridge 1 is in the first limit position A. In this position, the sensor 124 detects the permanent magnet 123 and switches off the fan 104 by means of control electronics 126 arranged on the printed circuit board 125. The first lever 120 is held in this switched-off position by a spring 127. In the second limit position B of the aroma cartridge 1, a second end 128 of the first lever 120 is actuated by the cover 58 arranged on the plunger 8 (cf. FIG. 5h). In the process, the spring 127 is then compressed.

The holder 111 for the aroma cartridge 1 interacts with a vibrating bridge 129 by way of the gearwheels 113 and 114. The vibrating bridge 129 (cf. also FIG. 5c) is suspended from the housing part 117b such that it can rotate about an axis of rotation D₁₂₉ and is designed in part as a toothed segment 130 and in part as a fork 131. The vibrating bridge 129 interacts with the gearwheels 113 and 114 via the toothed segments 130.

The vibrating bridge 129 and the holder 111 together form a second mechanical control means S₂, which is able to effect rotation of the aroma cartridge 1. Rotation of the vibrating bridge 129 about the axis of rotation D₁₂₉ in an arrow direction m is effected by means of the forks 131. As a result, rotation of the gearwheels 113 and 114 in an opposite arrow direction n about the axis of the rotation D₁₁₁is effected by way of the toothed segments 130, so that the holder 111 together with the aroma cartridge 1 rotates out of a basic position G in the direction of arrow n about the axis of rotation D₁₁₁ into an impregnation position T (cf. FIG. 5d). The second mechanical control means S₂ are actuated by third mechanical control means S₃. The third control means S₃ are formed by a switch mechanism 132. The switch mechanism 132 comprises a button 133, which is secured to the housing part 117b such that it can rotate about an axis of rotation D₁₃₃. The button 133 actuates numerous levers, which, as the third control means S₃, control the second control means S₂ and first control means S₁. The first control means S₁ substantially comprise a lifting fork 134, which is provided for the purpose of lifting the plunger 8. For this purpose, the lifting fork 134 engages beneath the cover 58 of the plunger 8 by way of lugs 135 (cf. also FIG. 5h).

FIG. 5b illustrates only the housing part 117b of the housing 102, which is shown in FIG. 5a. The second housing part 117b can be pivoted out of the first housing part 117a about axis of rotation D₁₁₇. If necessary, the two housing parts 117a and 117b can also be separated from one another. Exclusively mechanical components are arranged in the second housing part 117b. The electrical and electronic components are located exclusively in housing part 117a.

FIG. 5c illustrates FIG. 5b from arrow direction Vc. The control means S₁, S₂ and S₃ are constructed substantially symmetrically with respect to a center line M of the device 100. For example, the vibrating bridge 129 includes the two forks 131 and the two gearwheels 113 and 114, which are arranged mirror-symmetrically with respect to the center line M.

The following FIGS. 5d to 5g each illustrate only the control means S₁ or S₂ or S₃ which are of interest to the respective step of the sequence, in order to maintain clarity of the illustrations. FIGS. 5c and 5i in each case show all the control means S₁, and S₂ and S₃ which are visible in the respective view.

FIG. 5d shows part of the device 100 with the aroma cartridge 1 pivoted into an impregnation position T. The pivoting of the aroma cartridge 1 out of the basic position G illustrated in FIGS. 5a to 5c into the impregnation position T is effected by manually depressing the button 133, which in the process rotates about its axis of rotation D₁₃₃. As a result, control levers 136 which are fixed to the button 133 are moved in directions x′ and y′. Via bolts 137, the control levers 136 interact with tines 138 of the forks 131 of the vibrating bridge 129 and thereby rotate the vibrating bridge 129 in the direction of arrow m, so that the toothed segments 130 drive the gearwheels 113 and 114 (cf. FIG. 5c). With the gearwheels 113 and 114, the holder 111 together with the aroma cartridge 1 rotates through 180° in arrow direction n about axis of rotation D₁₁₁, so that the aroma cartridge 1 adopts the impregnation position T. The device 100 is now in what is referred to as a filling position II, having previously adopted at an at-rest position I (cf. FIGS. 5a to 5c).

For the following description, please refer in particular to FIGS. 5e and 5b. During rotation of the holder 111 with the aroma cartridge 1 into the filling position II, the lifting forks 134 are clamped and latched in order to retain the lifting forks 134 in a lower position L until the holder 111 together with the aroma cartridge 1 has rotated back into the basic position G of the aroma cartridge 1. FIG. 5e shows the lifting fork 134 in a clamped and latched position. The following sequence is intended for the clamping and latching of the lifting fork 134. The lifting fork 134 is initially in an upper position K and is mounted at the housing part 117b such that it can rotate about an axis of rotation D₁₃₄ (cf. FIG. 5b). Depressing button 133 causes a pressure fork 139, which is arranged in an articulated manner at the button 133 and grips a bolt 140 arranged fixedly on the lifting fork 134, to move substantially in arrow direction x′, thereby rotating the lifting fork 134 in an arrow direction o about its axis of rotation D₁₃₄. During this rotary movement about the axis of rotation D₁₃₄, a roller 141 mounted rotatably on the lifting fork 134 rolls down a limb 142 of a rocker 143. In the process, the rocker 143, which is mounted on the housing part 117b, rotates about an axis of rotation D₁₄₃ in an arrow direction p about the axis of rotation D₁₄₃. The rotary movement of the rocker 143 in the arrow direction p takes place counter to a spring leg 144, which is mounted at the button 133 such that it can pivot about an axis of rotation D₁₄₄. The spring leg 144 comprises a bolt 145 with a slot 146. A compression spring 147 is drawn onto the bolt 145. A web 148, which is fixedly connected to a second limb 149 of the rocker 143, is guided in the slot 146. During the rotation of the rocker 143 in arrow direction p, the web 148 compresses the compression spring 147 to an ever greater extent, thereby stressing the latter. Furthermore, a clamping fork 150 (cf. also FIG. 5c), which can pivot about an axis of rotation D₁₅₀, is also suspended from the housing part 117b. The clamping fork 150 has two tines 151, at which grippers 152 are arranged. Shortly before the button 133 has been fully depressed, bolts 153 which are formed integrally on the lifting fork 134 butt against contact surfaces 154 of the grippers 152 from above and press them in an arrow direction y′. In the process, a leaf spring 155 formed integrally at the clamping fork 150 is slightly prestressed against the pressure fork 139. This prestressing causes the grippers 152 to catch the bolts 153 of the lifting fork 134 with a rapid movement in arrow direction y as soon as the lifting fork 134 has reached its lower position L. The rapid movement of the clamping fork 150 in arrow direction y is effected by the stress of the leaf spring 155 being relieved when the bolts 153 slip over lugs 156 of the grippers 152. The holder 111 (not shown in FIG. 5e) together with the aroma cartridge 1 is now in the position II shown in FIG. 5d, and the impregnation body is impregnated, with the lifting fork prestressed, as shown in FIG. 5e. In this position II, the button 133 is fully depressed and is then either held down by the user or immediately released by the user. Despite the subsequent movement of the button in arrow direction x, the lifting fork 134 is held in its lower position L by the clamping fork 150 (cf. FIG. 5e). As a result, the rocker 143 and the spring leg 144 are also indirectly blocked. The stress in the compression spring 147 can also be relieved slightly as a result of the button 133 being raised back into its starting position, with the holder 111 together with the aroma cartridge 1 simultaneously rotating back (cf. FIG. 5f), but the compression spring 147 remains partially stressed by the web 148 which is indirectly blocked via the clamping fork 150. This means that the energy of the compression spring 147 is partially stored. The aroma cartridge 1, which together with the holder 111 forcefully rotates back out of the impregnation position T shown in FIG. 5d into the position shown in FIG. 5f, which is similar to the basic position G, butts against a web by way of the upper end of the guide element 39; this web connects two further tines 157 (cf. FIG. 5c—the web itself is covered) of the clamping fork 150. This butting action causes the bolts 153 to slip out of the grippers 152, thereby releasing the lifting fork 134. In the process, the clamping fork 150 rotates about its axis of rotation D₁₅₀. On account of the compression spring 147 acting on the web 148, so that it can release the stored energy, the rocker 143 is then rotated about its axis of rotation D₁₄₃ in arrow direction p′ and for its part causes the lifting fork 134 to rotate about its axis of rotation D₁₃₄ in arrow direction o′. In the process, the lugs 135 of the lifting fork 134 lift the plunger 8 at its cover 58, so that the plunger 8 is lifted out of its first limit position A into its second limit position or aroma-releasing position B (cf. FIGS. 5g to 5i, which show the device 100 in an aroma-releasing position III). In the aroma-releasing position III, the stress of the compression spring 147 is now fully relieved again (cf. FIG. 5g).

FIG. 5h once again shows a sectional side view of the housing 102 composed of housing parts 117a and 117b, with the device 100 in the aroma-releasing position III, in which the plunger 8 of the aroma cartridge 1 is in the second limit position B. When the plunger 8 is raised by the lugs 135 of the lifting fork 134, the cover 58 of the plunger 8 interacts with the lever 120 mounted at the bridge 121 and presses the second end 128 of the lever 120 in arrow direction x counter to the force of the spring 127. This causes the permanent magnet 123 arranged at the first end 122 of the lever 120 to pivot away from the sensor 124. Therefore, the sensor 124 provides the control electronics 126 with the information that the plunger 8 of the aroma cartridge 1 is in the aroma-dispensing position B. The fan 104 can then be controlled accordingly and can cause its air flow 110 to act on the impregnation body 21.

FIG. 5i shows an illustration of FIG. 5g from arrow direction Vi. This view shows the two lugs 135 of the lifting fork 134, which raise the plunger 8 at its cover 58.

Finally, FIG. 5j shows an illustration, corresponding to FIG. 5b, of the housing part 117b of the device 100. In this figure, the at-rest position I of the device 100, the upper position K of the lifting fork 134, the first limit position A of the plunger 8 and the basic position G of the aroma cartridge 1 have been reached again. The switching-off of the aroma cartridge 1 or the displacement of the plunger 8 out of position B into position A is effected by the button 133 being depressed slightly (cf. FIGS. 5c, 5g to 5i). In the process, the web arranged between the further tines 157 of the clamping fork 150 presses on the guide element 39 of the aroma cartridge 1 or the closure cap 25 and as a result slides the cover 58 off the lugs 135 of the lifting fork 134. As soon as the cover 58 slides off the lugs 135, the plunger 8 is retracted into the vessel 2 by the spring element 17. This then also causes the lever 120 to spring back into the position shown in FIG. 5a, in which the permanent magnet 123 is positioned opposite the sensor 124. In the process, the holder 111 together with the aroma cartridge 1 rotates slightly in arrow direction n about the axis of rotation D₁₁₁ until the cover 58 slips off the lugs 135 and then swings back into the basic position G in arrow direction n′ (cf. in particular FIG. 5h). The slight depression of the button 133 can be continued by deep depression of the button 133, so that the device 100 adopts the filling position II, in which the aroma cartridge 1 is impregnated and the sequence, which has already been described, commences.

To summarize, with regard to the control means S₁, S₂ and S₃, the following statements can be made in connection with FIGS. 5a to 5j: the first control means S₁, which are responsible for raising the plunger 8, are formed by the lifting fork 134 (cf. for example FIG. 5h). The holder 111 and the vibrating bridge 129 (cf. for example FIG. 5d) serve as second control means S₂, which are responsible for pivoting the aroma cartridge 1. The switching mechanism 132, which substantially comprises the button 133, the lifting fork 134, the control lever 136, the pressure fork 139, the rocker 143, the spring leg 144 and the clamping fork 150 (cf. for example FIGS. 5a, 5d and 5e), serves as third control means S₃, which control the first and second control means S₂ and S₃.

FIG. 6 shows a diagrammatic view of a further device 100 in which an aroma cartridge 1 is arranged. First control means S₁ are designed as electromechanical control means 158, which by means of an electric motor move an arm 159 in arrow directions x and x′ in order to extend a plunger 8 out of the aroma cartridge 1 and slide it back into the cartridge. The first control means S₁ are connected to third control means S₃ via a control line L₁. The third control means S₃ are designed as electronic control means 160 which control the first control means S₁ by means of electronics. Furthermore, the third control means S₃ control a fan 104 via a control line Lᵥ. The third control means S₃ receive information about the position of the plunger 8 from a sensor 124 via a control line Lₛ. When using an aroma cartridge 1, which in principle corresponds to the aroma cartridge described in FIGS. 2a to 2c, it is possible to dispense with second control means S₂, which pivot the aroma cartridge 1 by means of an electric motor serving as electromechanical control means 161. The second control means S₂ are connected to the third, higher-level control means S₃ by way of a control line L₂.

The invention is not restricted to exemplary embodiments that have been illustrated or described. Rather, it also encompasses refinements to the invention within the scope of the claims. In particular, the invention also provides for power to be supplied to the fan and also any further electrical or electronic components by means of a mains fitting or (storage) batteries and/or solar cells. A further variant embodiment provides for the device to be equipped with a wind-up fan, making it possible to produce a device which can be operated without electrical energy.

List of Reference Symbols:

1 Aroma cartridge
2 Vessel
3 Cavity, interior of 2
4 Aroma medium, liquid
5 Opening in 2
6 Environment
7 Metering device
8 Plunger
9, 10 End-side end of 8
11, 12 First, second seal
13 Sealing disk, sealing ring
14, 15 Stop of 8
16 Sliding guide
17 Spring element, compression spring
18 Inner wall of 2
19 Outer wall of 2
20 Annular recess for 17 in 2
21 Material for taking up 4, impregnation body
22 Base of 2
23 Grid at 8
24 Cage at 8
25 Closure cap
26 Neck of 2
27 Cylindrical passage in 25
28 Plunger ring seal at 8
29 Top side or bearing surface of 25
30 Underside or bearing surface of 25
31 Pocket at 14
32, 33 Journal at 2
34 Flattened section at 32
35 Cover for 25
36 External screw thread of 26
37 Internal screw thread of 25
38 Clip on 25
39 Guide element on 25
40 Web at 44
41, 42 Plate at 44
43 Annular shoulder of 25
44 Lifting tube
45 Cap for 44
46 Pin guided in 44
47 Cylindrical interior of 44
48 First mushroom-shaped end of 46
49 First abutment for 17 at 46
50 Second abutment for 17 at 45
51 Second end of 46
52 Basket
53 Projection at 25
54 Arm of 52
55 Ring of 52
56 Seal between 2 and 25
57 Gap
58 Cover of 44
59 Screw thread at 25
100 Device
101 Lever for engagement in 31
102 Housing
103 Rotary button
104 Fan
105, 106 Limb at 102
107, 108 Bore in 105 or 106
109 Interior of 100
110 Air flow of 104
111 Holder for 1
112 Arm of 111
113, 114 Gearwheel at 111
115, 116 Journal of 113 or 114
117a, 117b First or second housing part of 102
118 Rear side of 102
119 Front side of 102
120 First lever
121 Bridge
122 First end of 120
123 Permanent magnet at 120
124 Sensor
125 Printed circuit board
126 Control electronics
127 Spring at 120
128 Second end of 120
129 Vibrating bridge
130 Toothed segment of 129
131 Fork of 129
132 Switching mechanism
133 Button
134 Lifting fork
135 Lug of 134
136 Control lever
137 Bolt at 136
138 Tine of 131
139 Pressure fork
140 Bolt at 134
141 Roller rotatably mounted at 134
142 Limb of 143
143 Rocker
144 Spring leg
145 Bolt of 144
146 Slot in 145
147 Compression spring of 144
148 Web of 149
149 Second limb of 143
150 Clamping fork
151 Tine of 150
152 Gripper of 150 at 151
153 Bolt at 134
154 Contact surface of 152
155 Leaf spring at 150
156 Lug at 150
157 Further tine at 150
158 Electromechanical control means
159 Arm
160 Electronic control means
161 Electromechanical control means
A First limit position, impregnation position of 8
B Second limit position, odor-dispensing position of 8
D₂ Axis of rotation of 2 or 33, 34 or 1
D₁₁₁ Axis of rotation of 111
D₁₁₇ Axis of rotation of 117
D₁₂₀ Axis of rotation of 120
D₁₂₉ Axis of rotation of 129
D₁₃₄ Axis of rotation of 134
D₁₄₃ Axis of rotation of 143
D₁₄₄ Axis of rotation of 144
D₁₅₀ Axis of rotation of 150
F Force for extending 8
G Basic position of 1
K Upper position of 134
L Lower position of 134
L₁ -L₃ Control lines
Lₛ Control lines between 124 and S₃
Lᵥ Control lines between 104 and S₃
M Center line of 100
N_{g} Level of 4 in position G
Nₜ Level of 4 in position T
S₁ First control means
S₂ Second control means
S₃ Third control means
T Impregnation position of 1
Z Intermediate position of 8
d₁₁, d₁₂ Diameter of 11 or 12
d₂₁ Diameter of 21
h₈ Displacement of 8
l₈ Length of 8
m Arrow direction
n, n′ Arrow direction
o, o′ Arrow direction
p, p′ Arrow direction
x, x′ Arrow direction
I At-rest position of 100
II Filling position of 100
III Aroma-releasing position of 100

1. An aroma cartridge for a device for dispensing aroma substances, the aroma cartridge comprising at least one vessel having at least one cavity for receiving an aroma medium, the cavity having at least one opening, the opening being closed off by a metering device comprising a plunger that is displaced through the opening into two opposite limit positions, wherein the plunger, in the first limit position, is at least partially in an impregnation position within the vessel, and the plunger, in the second limit position, is at least partially in an aroma-dispensing position outside the vessel.

2. The aroma cartridge as claimed in claim 1, wherein the plunger includes at least one seal.

3. The aroma cartridge as claimed in claim 1, wherein the plunger includes two seals.

4. The aroma cartridge as claimed in claim 1, further comprising a first seal sealing off the cavity with respect to a surrounding environment in the impregnation position of the plunger, and a second seal sealing off the cavity with respect to the environment in the aroma position of the plunger.

5. The aroma cartridge as claimed in claim 1, further comprising a first seal sealing off the cavity from outside the vessel and a second seal sealing off the cavity from inside the vessel.

6. The aroma cartridge as claimed in claim 1, wherein a portion of the plunger has the ability to temporarily take up aroma medium.

7. The aroma cartridge as claimed in claim 1, wherein a portion of the plunger is a cage containing a material that is suitable for storing the aroma medium.

8. The aroma cartridge as claimed in claim 1, further comprising at least one spring element that presses the plunger into the first limit position.

9. The aroma cartridge as claimed in claim 1, wherein the metering device comprises a closure cap in which the plunger is mounted.

10. The aroma cartridge as claimed in claim 9, wherein the closure cap is screwed onto the vessel.

11. The aroma cartridge as claimed in claim 9, wherein the closure cap has at least one guide element at which the plunger is guided.

12. The aroma cartridge as claimed in claim 11, wherein the guide element is one of a rail and a bar.

13. The aroma cartridge as claimed in claim 1, wherein the vessel is a glass bottle.

14. The aroma cartridge as claimed in claim 1, further comprising at least one of a projection and a recess in at least one of the vessel and the metering apparatus.

15. The aroma cartridge as claimed in claim 1, wherein the plunger is assembled from at least two partial plungers.

16. The aroma cartridge as claimed in claim 2, wherein the seal is a sealing disk.

17. The aroma cartridge as claimed in claim 2, wherein the seal is a plunger ring seal.

18. The aroma cartridge as claimed in claim 2, wherein the seal is in gland form.

19. The aroma cartridge as claimed in claim 1, wherein the plunger has at least one stop.

20. A device for dispensing aroma substances, comprising the aroma cartridge of claim 1, a holder for releasably securing the aroma cartridge, and first control means for moving the plunger of the aroma cartridge into its limit positions.

21. The device as claimed in claim 20, further comprising second control means for pivoting the aroma cartridge in order to be impregnated.

22. The device as claimed in claim 21, further comprising third control means for controlling at least one of said first control means and said second control means.

23. The device as claimed in claim 22, wherein at least one of said first, second, and third control means comprise at least one of mechanical control means, electromechanical control means, and electronic control means.

24. The device as claimed in claim 22, wherein at least one of said first, second, and third control means are regulated by at least one of an electronic regulation system and an electronic regulation system assigned to the control means.

25. The apparatus as claimed in claim 24, wherein the regulation system comprises at least one sensor.

26. The device as claimed in claim 20, further comprising at least one fan, by which the plunger or the impregnation body is exposed to a flow of air in the aroma-dispensing position.

27. The device as claimed in claim 26, wherein at least one of the power of the fan and its air flow is regulated.

28. A method for controlling or regulating a device for dispensing aroma substances, said device comprising an aroma cartridge including a moveable plunger and a material which is able to absorb liquid, said method comprising the following steps:
rotating the aroma cartridge out of an upright basic position through approximately 90° to 180° into a loading position, to move the plunger of the aroma cartridge to a first limit position;
holding the aroma cartridge in the loading position in order to effect impregnation of the material which is able to absorb liquid;
rotating the aroma cartridge back into the basic position;
raising the plunger into a second limit position;
directing an air flow onto the plunger in order to dispense aroma substances; and
moving the plunger back into the first limit position.

29. A method for controlling or regulating a device for dispensing aroma substances, said device comprising an aroma cartridge including a moveable plunger, said method comprising the following steps:
raising the plunger out of a first limit position into a second limit position;
directing an air flow onto the plunger in order to dispense aroma substances;
moving the plunger back into a first limit position.

30. The device as claimed in claim 20, wherein said first control means comprises at least one of mechanical control means, electromechanical control means, and electronic control means.

31. The device as claimed in claim 20, wherein said first control means is regulated by at least one of an electronic regulation system and an electronic regulation system assigned to the control means.

32. The device as claimed in claim 20, wherein each of said first control means and said second control means comprises at least one of mechanical control means, electromechanical control means and electronic control means.

33. The device as claimed in claim 20, wherein each of said first control means and said second control means is regulated by at least one of an electronic regulation system and an electronic regulation system assigned to the control means.
